(19) United States
(12) Patent Application Publication
(10) Pub. No.: US 2021/0060240 A1
(43) Pub. Date: Mar. 4, 2021

(54) EXTENDED USE INFUSION SET FOR MEDICAL USE

(71) Applicants: Mark H. Scott, San Diego, CA (US); Leif Bowman, San Diego, CA (US); Nicholas John Foley, Edinburgh (GB)

(72) Inventors: Mark H. Scott, San Diego, CA (US); Leif Bowman, San Diego, CA (US); Nicholas John Foley, Edinburgh (GB)

(21) Appl. No.: 16/560,315

(22) Filed: Sep. 4, 2019

(51) Int. Cl. A61M 5/162; A61M 5/158; A61M 5/172

(52) U.S. Cl. CPC A61M 5/162; A61M 5/158; A61M 2205/3303; A61M 2005/1587; A61M 5/1723

(57) ABSTRACT

Briefly, an extended use infusion set is provided that substantially extends the therapeutic life of an infusion set, thereby increasing patient comfort, decreasing costs, and providing for increased efficacy for infused medicines and therapeutics. Generally, the extended use infusion set uses an insertion mechanism to position a cannula or needle at an initial depth and location for subcutaneous infusion of medication. At a later time, when the therapeutic effect of the medication has decreased below a threshold, the patient uses a reset mechanism to reposition the needle or cannula to a new depth and location. In this way, the therapeutic effect of the medication is reestablished, enabling the infusion set to be used effectively for a longer period of time. The infusion set also contemplates setting and resetting a biosensor.

### RELATED APPLICATIONS

This application claims priority to U.S. provisional application No. 62/726,715, filed Sep. 4, 2018, and entitled “Extended Use Infusion Set for Medical Use,” which is incorporated herein in its entirety.

### FIELD OF THE INVENTION

This invention relates to infusion sets for delivering therapeutics and medicines into the body of a mammal, including humans. More particularly, the invention relates to structures and processes for inserting a cannula or needle subcutaneously using a setting mechanism to obtain an extended therapeutic use of the cannula or needle.

### BACKGROUND OF THE INVENTION

Millions of people in the United States and around the world suffer from diseases that require the infusion of medicines, therapeutics, or biologics over extended periods of time. For example, millions of people just in the United States suffer from diabetes and require insulin continually to just stay alive and have an opportunity for a normal life. For some, regular injections are sufficient, however many may benefit from continuous monitoring and delivery of insulin. These people wear a continuous glucose monitor for monitoring their glucose levels, which provides information which can drive or can inform the user to adjust a pump that delivers insulin into their system through an infusion set. It may also suggest they inject a bolus (larger single dose) or that they should eat. In this way, the patient can maintain a more constant blood glucose level, avoid dangerous highs and lows in glucose levels, and have the best opportunity for a normal and comfortable life. Further, many people who currently use needle injections could benefit from a more continuous treatment that can be provided through continuous glucose monitoring, pumps, and infusion sets. However, due to their cost, inconvenience, and pain, many people who could benefit do not use continuous glucose monitors, infusion pumps, and infusion sets.

Although the disclosures herein regarding infusion sets will focus on using infusion sets for primarily insulin delivery, it will be understood that similar benefits may be achieved for other types of medicines, therapeutics, and biologics.

Current infusion sets suffer from several deficiencies. Most often, a patient is trained to attach the infusion set to their body. Often this can be an involved and complicated process, and often the patient must try several times to get infusion set to be properly attached for delivering medicine. This can be painful, and also expensive, as sometimes multiple attempts will destroy the infusion set.

Once installed and in operation, infusion sets typically have a duration of only 1-3 days before it is necessary to remove the infusion set and find a new location on the body. Since infusion sets are designed for a single use, each time the infusion set is removed, the patient is required to attach a new infusion set, which may also mean a new purchase, although infusion sets are often available in monthly supply kits. The result is patients needing between 100+ to 300+ disposable infusion sets annually. In this way, the decision to move from injection-based insulin to continuous-based insulin delivery can involve a significant and oftentimes prohibitive expense, despite its therapeutic benefits to the patient.

Unfortunately, current infusion sets provide substantially reduced therapeutic benefit after the first 24 hours. That is, after 24 hours, the effectiveness of the medication delivery is reduced, thereby requiring an increased dosage of the medication, or potentially having the patient not receive a full therapeutic and beneficial effect. Many factors affect how long an infusion set will give therapeutic effect. For example, whether or not the cannula was properly installed, the depth that the cannula was installed under the skin, whether the cannula was or became damaged or kinked under the skin or outside of the body, and the physical activity of the patient. Further, it has been found that the tissue changes at the point in which medicine is delivered and becomes resistant to further delivery. There may be hardening of the tissue around the end of the needle or cannula, biological responses such as macrophages, crystallization of the infusion fluid in or around the infusion cannula, or other biological changes such that the backpressure on the fluid increases. There may be changes in the fluid absorption capacity of the tissue at the position of greatest fluid transfer, even without a sensation that may be described as hardening, that decrease the effectiveness of the fluid medicine to achieve its therapeutic benefits.

An infusion set delivers medicine into the body through a cannula that is essentially a thin flexible tube for delivering medicine to a particular depth and position under the skin. However, this cannula is too soft and flexible to penetrate the skin and be pushed to the therapeutic location. Accordingly, infusion sets use an introducer needle that extends through the cannula and is used to pierce the skin and forge the path through patient tissue to the therapeutic location. Once the cannula is in a proper position, the introducer needle is removed and discarded, along with any insertion mechanism. In some cases, the insertion mechanism is reusable instead of disposable.

The current state of the art for infusion sets uses an introducer needle with a soft cannula around it for the length of the needle that penetrates the skin. The end of the soft cannula is formed onto the surface of the introducer needle to ensure a tight fit that can withstand the forces of insertion through the skin. As the introducer needle usually has a beveled edge to create a sharp point or edge to penetrate the skin, it is necessary for the introducer needle to protrude approximately 3 mm beyond the end of the soft cannula. The result is that a patient who requires a soft cannula tip to deliver medication into the body at a depth of 6 mm must insert the introducer need 9 mm deep. Such a deep insertion creates a deeper wound and more pain than necessary and is additionally intimidating to the user.

Further, the introducer needles used in the current infusion sets are seen by the user upon removal, and this view of the needle is intimidating to the user and reinforces the negative aspect of inserting infusion sets.

### SUMMARY

An extended use infusion set is disclosed that substantially extends the therapeutic life of an infusion set, thereby increasing patient comfort and convenience, reducing patient intimidation, decreasing costs, and providing for increased efficacy for infused medicines and therapeutics. Generally, the extended use infusion set uses an insertion mechanism to position a cannula or needle at an initial depth and location for subcutaneous infusion of medication. At a later time, when the therapeutic effect of the medication has decreased below a threshold, or the patient has discomfort, the patient uses a reset mechanism to reposition the needle or cannula to a new depth and location. In this way, the therapeutic effect of the medication is reestablished, enabling the infusion set to be used effectively for a longer period of time.

In one example, the extended use infusion set has a base that is attached to the skin of a patient with an adhesive pad. The base also has a reset mechanism that receives a head portion of the cannula or needle. A setting mechanism sets the head portion at an initial position in relation to the reset mechanism. This sets the cannula or needle at its initial depth and location. At a later time, the patient moves the head portion in relation to the reset mechanism, which causes the needle or cannula to be positioned at a new depth and position. The reset mechanism may allow the head portion to slide, snap, rotate, or otherwise be moved by the patient into a second position, or subsequent positions. In some cases, the reset mechanism may allow for only a one-dimensional reset, and in other cases may allow for two dimensional or even three-dimensional movements. In this way the reset location can be limited by the particular structure of the reset mechanism.

Advantageously, the extended use infusion set has a substantially increased useful life over known infusion sets. By extending the life of the infusion set, patient comfort is increased, as the patient can reestablish full therapeutic effect of their existing infusion set without the need to find a new insertion location, and go through the discomfort of the full needle insertion process. Further, since the insertion set can be used for an extended period of time, the number of insertion sets that the patient needs to use in a year is substantially reduced. For example, if the extended use infusion set doubles the useful life of the infusion set, then the cost for infusion sets is potentially cut in half for the patient. Not only does this save the patient or healthcare system money, but a reduced cost would allow more patients to benefit from continuous infusion. Further, since the extended use infusion set enables simplified reestablishment of full therapeutic effect, patients and doctors are more likely to keep the patient at a more effective level and more constant therapeutic value. In this way, the medication will have a more consistent effect on the patient, thereby improving the quality of life and medical results for the patient.

The extended use infusion set may also be advantageously used with biosensors, such as sensors for detecting blood glucose levels. Similar to setting a cannula, the extended use infusion set may be used to set and reset a biosensor for extended sensing. In some cases, a biosensor and cannula may be used at the same time, for example through two available ports, and other times the biosensor may be used to replace the cannula.

### BRIEF DESCRIPTION OF THE DRAWINGS

These and other objects and advantages of the invention will become apparent upon reading the following detailed description and upon referring to the drawings and claims.

FIG. 1 has illustrations of a top view, a side view and a picture showing an extended use infusion set in accordance with the present invention.

FIG. 1A have picture illustration views showing an extended use infusion set in accordance with the present invention.

FIG. 2 has illustrations of a top view and a side view showing an extended use infusion set in accordance with the present invention.

FIG. 3 has illustrations of a top view and a side view showing an extended use infusion set in accordance with the present invention.

FIG. 4A has an illustration of a side view showing an extended use infusion set in accordance with the present invention.

FIG. 4B has an illustration of a side view showing an extended use infusion set in accordance with the present invention

FIG. 5 has an illustration of a side view showing an extended use infusion set in operation and in accordance with the present invention.

FIG. 6 has illustrations of a partial cut-away front view showing an extended use infusion set in operation and in accordance with the present invention.

FIG. 7 has illustrations of a partial 3-D view showing an extended use infusion set in operation and in accordance with the present invention.

FIG. 8 has illustrations of a partial 3-D view showing an extended use infusion set in operation and in accordance with the present invention.

FIGS. 9A, 9B and 9C are partial 3-D views of an extended use infusion set in operation and in accordance with the present invention.

FIG. 10 is an illustration of a top view of an extended use infusion set in accordance with the present invention.

While the invention will be described in conjunction with example embodiments, it will be understood that it is not intended to limit the invention to such embodiments. On the contrary, it is intended to cover all alternatives, modifications and equivalents as may be included within the spirit and scope of the invention.

### DETAILED DESCRIPTION OF THE EXEMPLARY EMBODIMENTS

The embodiments and examples shown here are to provide enough information to fully understand the invention. One skilled in the art will understand how minor changes or deviations can be made and still be within the scope of the invention. The following description of exemplary embodiments of the invention is not intended to limit the scope of the invention to these exemplary embodiments, but rather to enable any person skilled in the art to make and use the invention. To assist in a clear and unambiguous understanding of the disclosure, the following definitions are used:

### Definitions

A bolus is generally a single dose of insulin, typically a larger dose administered at once and often at mealtime. The purpose is to provide additional insulin to help the body address the carbohydrate intake of the meal. A bolus may also be given if there are other foreseen or unforeseen events affecting blood sugar. A bolus may be administered via an infusion set which also delivers basal insulin, or may be injected separately.
The basal insulin level is the amount of insulin referred to as necessary to address the diabetic patient's need for insulin between meals or other foreseen or unforeseen events, such as mealtime or snacks. The basal insulin dosage is often a lower level, delivered more regularly or nearly continuously, between meals.
A cannula is similar to a hollow metal needle, but often made of a softer, flexible material which can bend. Some cannula designs incorporate an introducer needle, which may or may not be hollow, to help the cannula achieve its desired position.
An introducer needle is a hollow or solid needle which generally does not deliver medicine itself, but instead helps the cannula penetrate the skin and achieve its desired position to deliver medicine for therapeutic benefit at a subdermal or subcutaneous depth and position.
A needle is a sharp object which is used to penetrate the skin and push through the body to a desired depth or position. A needle may be hollow or solid. It may allow medicine to travel through it into the body, or it may act only as an introducer needle for a softer and/or more flexible cannula and be retracted after its positioning function, or it may perform both medicine delivery and introducer functions.
A depot is a location where medicine is delivered by the needle or cannula, and generally the “depth and location” within the patient tissue where the medicine is absorbed by the body.

Embodiments of the present invention are directed to a medical infusion set constructed to insert a flexible cannula to an initial therapeutic depth and position under the skin of a patient. Herein, the term “depot” may be used to identify a particular depth and position for a subdermal or subcutaneous insertion of a cannula. At a later time after, such as 24 hours after the initial insertion, or when the initial insertion has a measured degraded therapeutic effect, the insertion set is capable of repositioning the cannula to a new therapeutic depth or position. In this way, therapeutic effect may be greatly increased at the new depth and position site, enabling an extended use of the insertion set.

A particular embodiment is directed to an infusion set with the capability to have an initial insertion to a therapeutic depth and position of a curved needle, which may thereafter be further inserted or partially removed for the purposes of achieving a new depot.

A preferred embodiment is directed to an infusion set with the capability to have an initial insertion to an effective depth and position of a sensor such as for a continuous glucose monitor (CGM), which may thereafter be further inserted or partially removed for the purposes of achieving a new depth and/or position at which interaction with biological processes can provide useful data.

A preferred embodiment is directed to an infusion set with the capability to have an insertion to an effective depth and position of a sensor such as for a continuous glucose monitor (CGM) for the purposes of interaction with biological processes that can provide useful data.

As shown in the included figures, the illustrations depict instances of infusion sets inserted into the skin for the purposes of delivering fluid medicine into the tissue beneath the outer layer of skin, such as insulin for the treatment of diabetes subcutaneously. However, it will be understood that the invention may also be utilized for delivery of other medicine, hormones, vitamins, saline, including fluids containing dissolved or suspended solids if in the future such a treatment is created. The invention may be used for the placement of sensors capable of measuring biological information, such as glucose levels, ketone levels, lactate levels, salinity, red or white blood cells, T-cell counts, dissolved oxygen, or the like on a continuous or intermittent basis, whether for information, entertainment, or compliance purposes only, as part of a feedback loop in medicine delivery, or to aid in a combination of manual and automated administration of fluid described above, whether that manual administration is through the infusion set or administered elsewhere on the body.

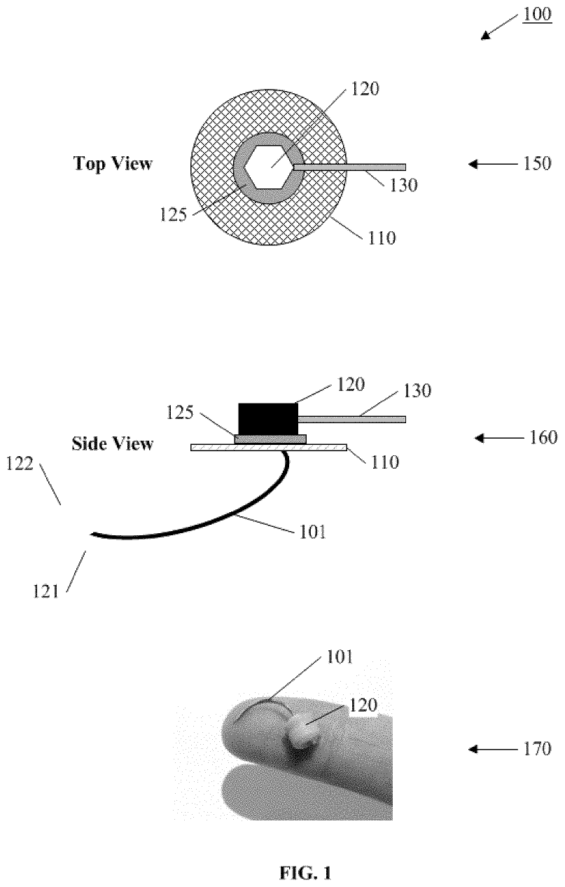

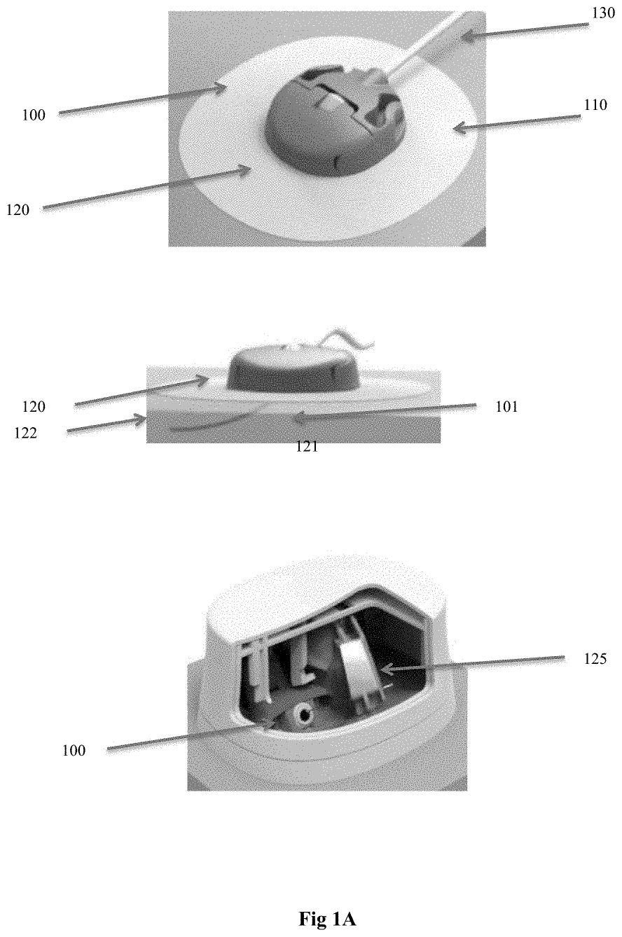

Referring now to FIG. 1 and to FIG. 1A, an infusion set 100 is illustrated. Infusion set 100 is illustrated in a top view 150 as well as a side view 160. Infusion set 100 is designed and intended to insert a curved cannula 101 subcutaneously into a human to enable a medicine or therapeutic to be delivered into the patient at a desirable depth and position. As illustrated, the infusion set 100 has already been inserted under the patient's skin to an initial depth and position. It will be understood that a housing, mechanism, or introducer needle may have been used to insert and set the infusion set 100 into the illustrated position. Example insertion mechanisms are illustrated in following sections.

The infusion set 100 is attached and secured to the human body using an adhesive pad 110. A head piece 120 connects tubing 130 to the source of the medication, which in some cases may be insulin driven from an infusion pump. Head piece 120 also connects to the cannula 101. Cannula 101 may be made from metal, plastics, or other materials appropriate for delivery of medication. It will be understood that the selection of material for cannula 101 may be particularly selected for the type of medication to be delivered. For example, some chemotherapy medications may degrade certain materials, so materials resistant to chemical damage would need to be selected. It will also be understood that the cannula 101 may use an introducer needle to initially place the cannula 101 to its desired position or location 121, with the introducer needle then being removed and discarded.

The headpiece 120 is used to insert the cannula 101 to and initial depth and position 121. Once inserted and properly adhered to the human body, the infusion pump can inject medication or therapeutic through tube 130 and into cannula 101, which delivers the medication at or near the initial depth and position 121. As is known, the therapeutic effect of the delivered medication will reduce over time, and typically the cannula would need to be removed and a new infusion set used. In many cases, the therapeutic effect remains quite high for the first 24 hours, and then begins to degrade over the next two or three days. Often, the infusion set would need to be changed after about 1 to 3 days, if not sooner.

To obtain an extended use, infusion set 100 has a reset mechanism 125 that can be used by the patient to extend the therapeutic effect using the same infusion set 100. In this way, infusion set 100 can have an extended life as compared to known infusion sets. A patient that is using an infusion set often has continuous or at least regular monitoring of therapeutic effect. In this way, the patient would become aware that the therapeutic effect of the medication has reduced over time, and the position of the cannula 101 needs to be changed. In other cases, the patient may begin to experience discomfort, and desire that the cannula be moved to a new depth or location. Accordingly, when the patient learns that therapeutic effect needs to be improved, or otherwise desires to move the cannula, the patient uses reset mechanism 125 to reset or reposition the cannula 101 to a new depth and position 122. In some cases, the new location may be at the same depth but a different position, in other cases only the depth will change, and in other cases both the depth and position can change.

It will be understood that reset mechanism 125 may be constructed to allow for only a single repositioning of cannula 101, or in some cases may be constructed to allow multiple repositioning. As will be understood, the decision on how many repositionings to allow may be made on a patient by patient basis, and can be influenced by the particular medication being infused, or the particular therapeutic effect that is desired. In another implementation, the repositioning may be initiated upon finding that glucose control is degrading or has become less effective. For example, a patient's mobile phone application may be in communication with his or her continuous glucose monitoring (CGM) device. The phone application will from time to time receive glucose and insulin data from the CGM device, and using algorithmic processes, may provide an alarm or notice to the patient that his or her glucose control is degrading. Upon receiving this notification, the patient may reset the position or depth of the cannula as discussed, resulting in improved glucose control. Using prior systems, the patient would be forced to make the decision either to accept the degraded performance for a longer period of time or to immediately endure the cost and pain of inserting a new infusion set at a new location.

Reset mechanism 125 may take several forms. For example, reset mechanism may take the form of a rotatable disk that upon rotation pushes the cannula 101 further into the patient, thereby moving the cannula from position 121 to position 122. It will be understood that the rotation may be done in a free-form manner, or may have hard stops that limit the possible change in insertion position and location. In another example, the reset mechanism 125 may be a snap receiver that allows the patient to press the head 120 into the reset mechanism 125 such that the cannula 101 is moved from position 120 to 122. In this way, the patient can simply press head 120 until it steps into a new, lower (closer to the skin) position in the reset mechanism 125. In another example, the reset mechanism 125 may be constructed to have a sliding track that would allow the user to reposition the head 120 latterly within the reset mechanism 125. Allowing the patient to latterly move the head 120 can reposition the cannula 101 from position 121 to position 122. The track may allow for free-form sliding, or may have stops or tabs for setting a more limited or precise positioning. It will also be understood that the tracks may be set to allow for one repositioning, or for multiple repositionings. It will be understood that many alternative mechanical structures can be used to reposition the cannula.

As described thus far, reset mechanism 125 is used to extend the cannula 101 either further or deeper into the patient, thereby repositioning the cannula into an area that has not yet received direct infusion of the medication. It will be appreciated that the reset mechanism 125 may also be constructed to retract the cannula 101, for example, by a few millimeters. In this regard, the head 120 may be rotated away from the reset mechanism to retract cannula 101; the head 120 may snap outward from the reset mechanism to allow the patient to pull the head 120 to a new position within the reset mechanism 125; or the head 122 may slide on a track that retracts the cannula 101. In this way, the cannula does not need to be constructed to reposition into new tissue but can be retracted into tissue that has already been pierced. It will be understood that many alternative mechanical structures can be used to reposition the cannula. Depending upon the specific construction of the cannula and reset mechanism, it may be possible to adjust only the depth, only the position, or both the position and depth.

Advantageously, the reset mechanism 125 enables the head 120 to be moved in a way that can reposition the cannula 101 to a new depth and position within the patient, thereby extending the time that the infusion set 100 may remain on the patient before replacement. Not only will this save discomfort and cost for the patient, but may also allow for a more consistent delivery of the therapeutic medication. For example, since the therapeutic value of the medication remains quite high for the first 24 hours, it may be possible to construct an infusion set 100 where the reset mechanism 125 allows for several small resets. In this way, the patient would be allowed, for example every 24 hours, to make a small repositioning of the cannula. In this way, the therapeutic effect of the medication can remain consistent and high. Although several mechanisms have been presented for the reset mechanism 125, it will be appreciated that there are several alternatives to providing a mechanical reset mechanism 125 for moving the position of cannula 101.

FIG. 1A shows one example of the cannula 101 connected to the head 120. As can be seen, the needle 101 is curved, and constructed of a metal material. It will be understood that the cannula 101 can be constructed from several different materials, including combinations of materials (e.g., metal and plastic/polymeric), and may be made with different curves, lengths, and thicknesses.

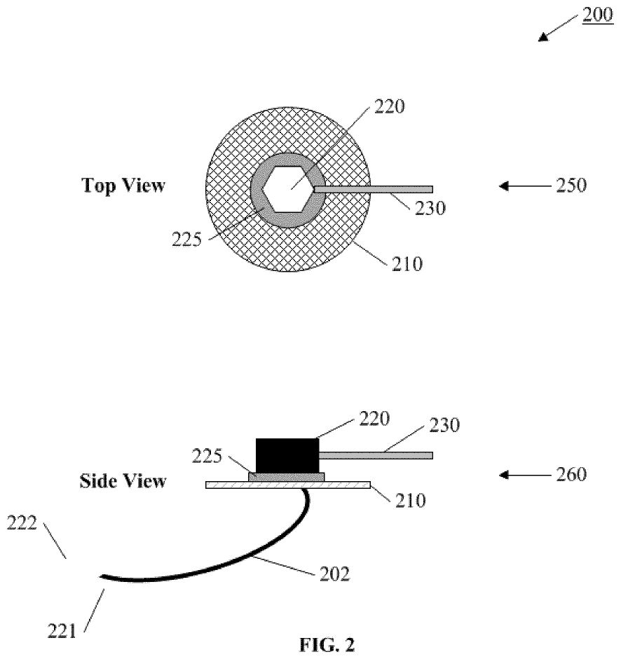

FIG. 2 illustrates an infusion set 200 showing a top view 250 and a sideview 260. Infusion set 200 is similar to infusion set 100 described with reference to FIG. 1, so will only be briefly discussed to illustrate the differences. Infusion set 200 is also adhered to the human body with adhesive contact 210 and has a head 220 which allows medication to be injected through a tube 230. The medication flows from tube 230 into a curved needle 202. The head 220, and possibly an associated housing and insertion mechanism, positions the curved needle 202 to an initial depth and location 221. The curved needle 202 will often be made of a metal material, however stiff plastics or other materials may be used. The curved needle 202 has a sufficiently hard and sharp distal end and such that it can pierce and move through the patient tissue to properly position the needle 202. Infusion set 200 also has a reset mechanism 225 that can be used by the patient to set the curved needle from its initial position at 221 to a new position 222. In some cases, the new location may be at the same depth but a different position, in other cases only the depth will change, and in other cases both the depth and position can change. It will be understood that the reset position may take many forms, such as a rotatable disk, sliding on tracks, or a vertical snap mechanism as previously discussed with reference to FIG. 1.

Infusion set 200 does not need a disposable introducer needle, as the curved needle 202 acts both as the introducer needle, and as it is hollow, can itself act as the cannula for delivering the therapeutic medication. In this way, waste is reduced, and the curved needle 202 may be readily repositioned into new tissue. Advantageously, the infusion set 200 enables the curved needle 202 to be repositioned one or multiple times to extend the usable life of the insertion set 200.

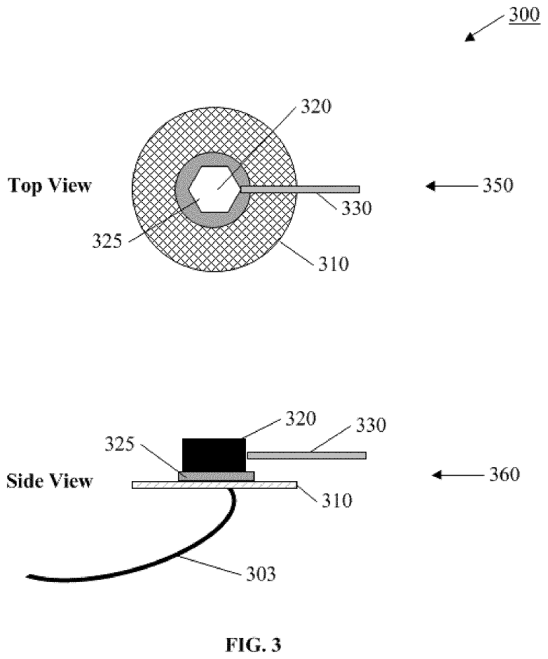

Referring now to FIG. 3, a biological sensor 300 is illustrated with a top view 350 and a side view 360. The biological sensor 300 is positioned adjacent the skin of a human body and with the use of an inserted sensor 303 is able to detect certain types of biological activity within the body. For example, the inserted sensor may be constructed to detect the level of glucose in blood. In another example, the inserted sensor may detect other interstitial and/or blood components that may be indicative of the health and well-being of the patient. It will be understood that inserted sensor 303 may be constructed to detect a wide variety of conditions.

The biological sensor 300 is attached to the body with the adhesive pad 310, and has a head 320 that is used to assist in insertion of the inserted sensor 303. In some cases the head 320 may also contain electronics that cooperate with the inserted sensor 303 for detecting blood components. The inserted sensor 303 may have a tip that is sufficiently hard and sharp to penetrate the human flesh. In other cases, an introducer needle may be used to initially position the inserted sensor 303, and then the introducer needle is removed and discarded. Biological sensor 300 in some cases may also be constructed with the ability to inject the therapeutic medication. In such a case where the sensing and infusion functions are combined (not illustrated), a tube 330 may be connected to a medication source, such as an infusion pump, for delivering the medication. Further, the head 320 may be constructed to communicate to other medical devices, such as devices to present biological information, or to send control information an infusion pump. It will be understood that the communication may be done either wired or wirelessly.

The biological sensor 300 also has a reset mechanism 325. The reset mechanism 325 is similar to the reset mechanism 125 discussed with reference to FIG. 1. Accordingly, the reset mechanism 325 enables the patient to reposition the inserted sensor 303 to a new location, thereby enabling a longer usable life for the biological sensor 300 or allowing the sensor to maintain optimal performance and avoid biological response processes known to impact sensor duration and/or performance. In this way, costs and patient discomfort can be reduced, while also increasing the reliability and life for the biological sensor 300. It will be understood that the reset mechanism may be used to change only the depth, only the position, or in some cases change both the position and depth of the biological sensor.

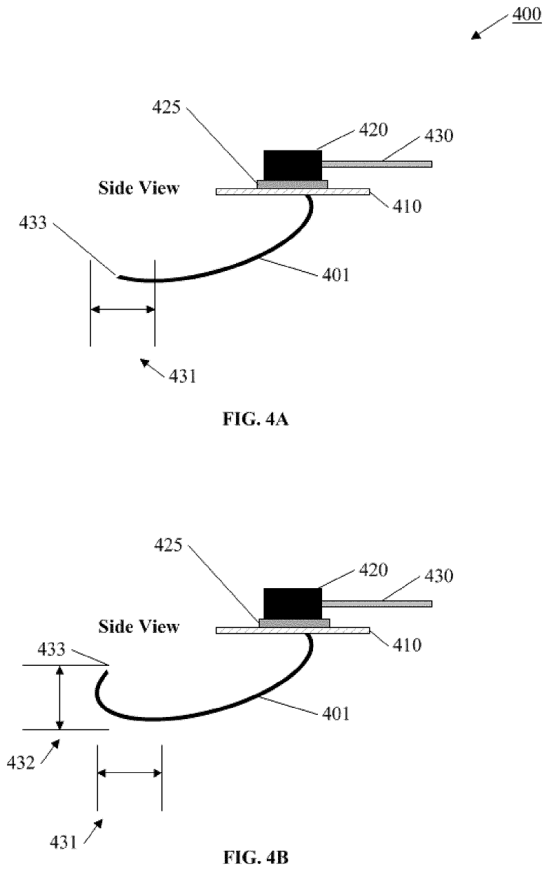

Referring now to FIG. 4A and FIG. 4B, an insertion set 400 is illustrated. Insertion set 400 is similar to insertion set 100 described with reference to FIG. 1. Insertion set 400 has a needle 401 that connects to a head piece 420 that can receive medication through tube 430 for delivering a therapeutic medicine to a patient subcutaneously. The insertion set 400 is attached to the human body using an adhesive pad 410 in some cases, the head piece 420 and needle 401 will be inserted using a housing or disposable insertion mechanism or a reusable insertion mechanism to initially position the tip of the needle 433 to an initial depth and position.

Reset mechanism 425 is constructed to allow movement of the headpiece 420 in more than one axis. For example, the reset mechanism 425 may be set such that it may be rotated so that the needle 401 moves in a circular or oblong path changing both an X and Y position simultaneously, such as a helical coil like a spring. In another example, the reset mechanism for the 25 may provide for a screw rotation that allows the head 420 to both move in the Z axis as well as in the X or Y axis. Depending on the particular construction for reset mechanism 425, the reset mechanism 425 can be constructed to cause the repositioning of the tip of the needle 433 in one dimension, two dimensions 431 or in three dimensions as shown by 431 and 433. Or stated differently, the reset mechanism may be constructed to allow for a resetting of depth, a resetting of position, or a resetting of both depth and position

In one particular example, the head 420 may rotate in the reset mechanism 425 so that the head 420 follows a sloped vertical guide that causes the head 420 to lift away from the skin or be pushed toward the skin depending upon the direction of rotation. At the same time, the needle 401 may be positioned off-center in the base of the head 420. In this way the same rotation motion within the reset mechanism causes the needle 401 to move in both the X and Y directions. In this way, the reset mechanism 425 causes a three-dimensional change in the position of the needle 401. In another example, the head 420 may be constructed to be rotatable and able to be snapped to adjust its vertical position on an alignment track. At the same time, the head 420 may be positioned on a sliding track that allows the head 420 to be positioned in an X, Y, or Z direction. The ability to steer the head insertion allows the design to specifically locate the head location in the body and relative to desired biological components or structures (e.g., proximity to specific blood vessels or capillary beds, including the specific side or orientation relative to them such as above or below).

It will be appreciated that by enabling reset mechanism 425 to act in 2 or more dimensions, more flexibility in repositioning is obtained. In one advantage, an individual patient may find through experience that a change in a particular dimension is more comfortable for them, or gives a better and longer therapeutic result. In another advantage, a multidimensional repositioning can allow for positioning of more sophisticated needle or cannula shapes, such as helical or other three-dimensional shaped infusion devices.

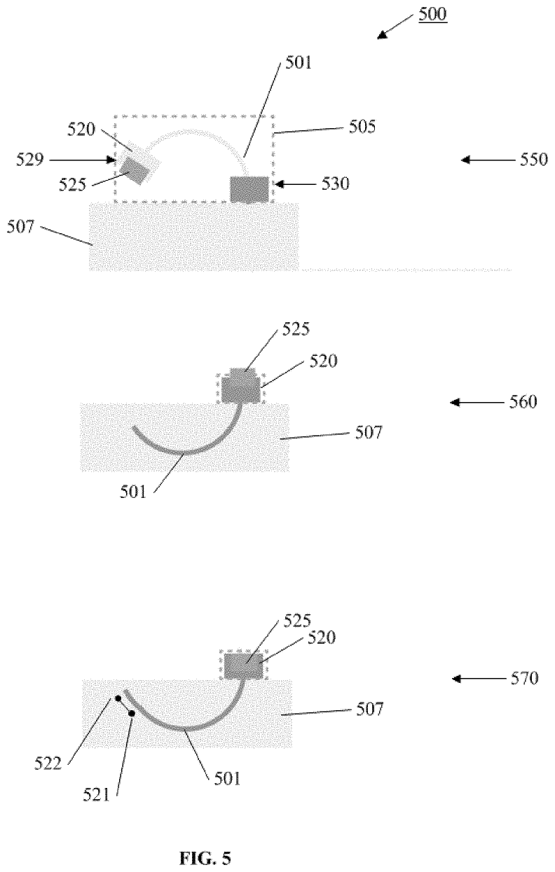

Referring now to FIG. 5, a specific example of an infusion set 500 is illustrated. In view 550, the infusion set 500 is illustrated with the head 520 coupled to cannula 501 inside of infusion housing 505. Infusion housing 505 is temporarily provided to assist in the positioning of the cannula 501 under the skin. After insertion of the cannula 501, the housing 505 can be fully or partially removed. More particularly, the housing and insertion mechanism is intended to move the head 520 from an initial position 529 to the final position 530. In this way, the cannula 501 pierces the skin 507 and enables the cannula 501 to be positioned at a therapeutic depth and position in the patient tissue 507.

View 560 shows the infusion set 500 with the cannula 501 positioned at its initial depth and location 521. The head 520 is positioned up against the patient skin, and is adhered to the skin using an adhesive pad 510. At this point, the infusion set 500 may be used from 1 to 7 plus days while maintaining sufficient therapeutic effect from the medication. It will be understood that the length of time it may be used will vary depending upon patient, medication used, and desired minimum effect. Reset mechanism 525 is also in its initial position, and has not yet been activated.

View 570 shows infusion set 500 after the reset mechanism 525 has been activated. Here, the reset mechanism is illustrated as a snap button that the patient presses further into head 520. Although a single step location is illustrated, it will be understood that several intermediate steps may be provided, thereby allowing for multiple resettings for depths, positions, or positions and depths. It will also be understood that the reset mechanism 525 may be in the form of a rotatable disk that is inserted through a screw-like mechanism. Once the reset mechanism 525 has been activated, the cannula 501 is moved from its initial depth and position 521 to a new repositioned depth and location 522. In this way, the therapeutic effect for the medication is improved or maintained. Although the reset mechanism 525 is illustrated as moving the cannula 501 further into the body, it will be understood that the reset mechanism 525 can be constructed such that the reset mechanism retracts the cannula 501 (e.g., a few millimeters). In this way, the cannula 501 would not need to be constructed to pierce or penetrate new tissue, but retracts into the initial existing wound site. It will also be understood that the infusion set can easily be modified to insert and reset a biological sensor, such as a sensor for glucose monitoring.

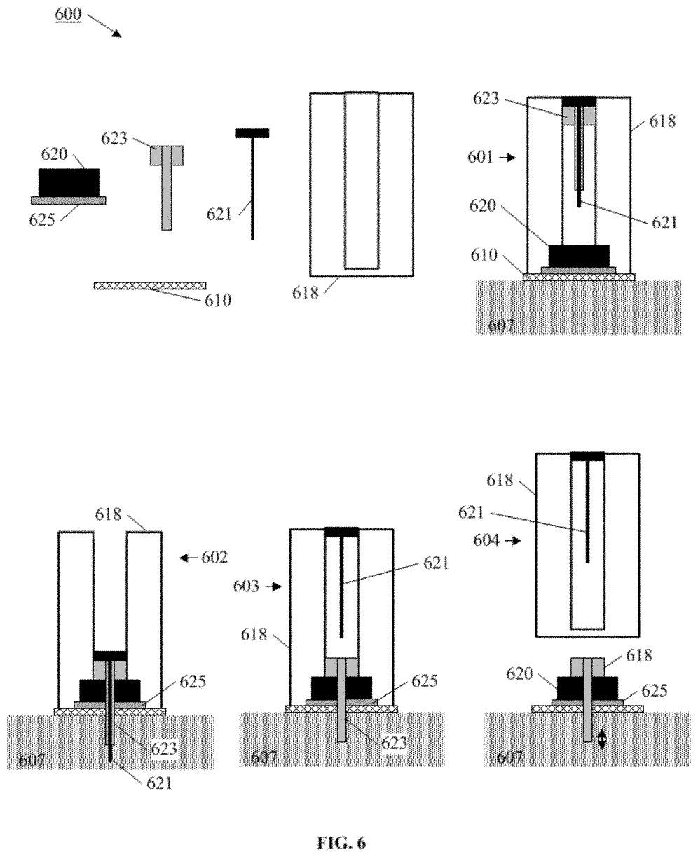

Referring now to FIG. 6, another infusion set 600 is illustrated. In view 601, infusion set 600 is illustrated prior to insertion and use. In this position, a housing 618 is used to assist with the insertion and initial placement, and after the initial insertion and placement, the housing 618 is discarded or set aside for later reuse, within sanitary guidelines. The housing 618 assists in adhering the infusion set 600 to the skin 610 using adhesive pad 612. The housing contains an introducer needle 621 in and providing a path for the cannula 623 to be positioned at its initial depth and location. The infusion set 600 also has a head piece 620 that cooperates with a reset mechanism 625 to provide for repositioning of the cannula 623 at a later time. It will be understood that the setting mechanism and cannula can be constructed and made to cooperate in a way that can change only the depth, change only the position, or change both the depth and position.

View 602 shows the housing 618 with the introducer needle 621 fully extended in two the patient's tissue 610. In this position, the cannula 623 is also positioned in to its initial depth and location. View 603 shows that the introducer needle 621 has been removed from the patient, while the cannula 623 remains in its initial position. It will be understood that removal of the introducer needle may be accomplished in several ways. For example the introduced needle may be removed as part of a manual motion by the patient to remove the insertion mechanism and housing. It will be appreciated that the introducer needle can be removed concurrent with removal of the housing or may be done prior to removing the housing. In one application, the introducer needle is removed by a spring or other tension device prior to removal of the housing. In this way, the removal of the introducer needle from the patient is independent of the action to remove the insertion mechanism and housing.

View 604 shows that the housing 618 and introducer needle 621 are removed and discarded or set aside for later reuse, within sanitary guidelines. The cannula 623, head 620, and reset mechanism 625 remain attached to the patients's skin. At a later time after the therapeutic effect of the medication has been reduced, then the patient can use the reset mechanism 625 to reset the depth or location of the cannula 623 as previously discussed. It will also be understood that the infusion set can easily be modified to insert and reset a biological sensor, such as a sensor for glucose monitoring.

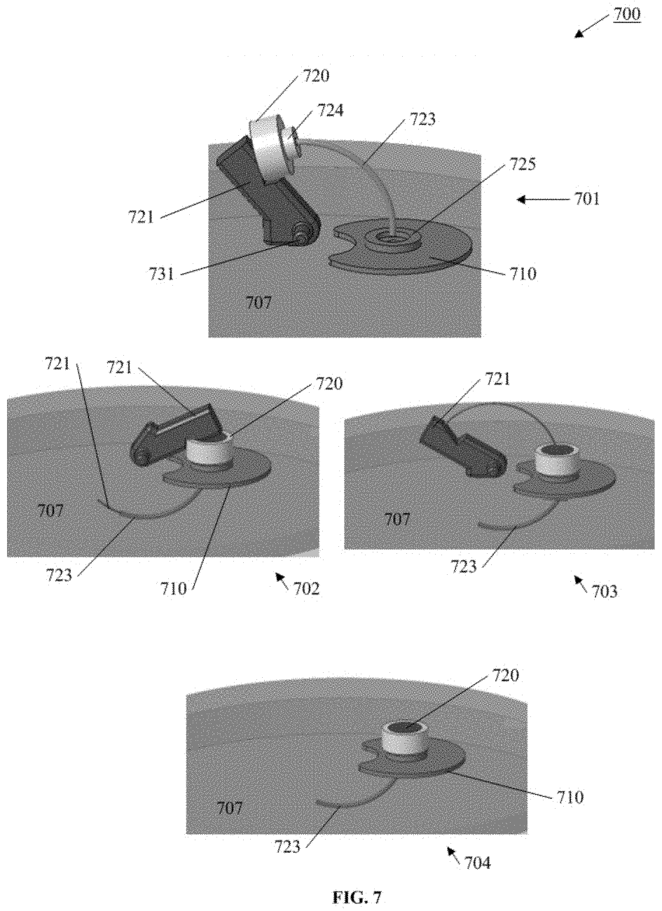

Referring now to FIG. 7, an infusion set 700 is illustrated. Infusion set 700 is constructed to require the use of an introducer needle 721 to first pierce the patient's skin and then position the cannula 723 into the tissue at its initial depth and location. The introducer needle 721 is illustrated as a two-part device. First, the introducer needle has a large insertion tab that is hinged 731 and connected to a housing (not illustrated). The needle portion of the introducer needle 721 is positioned inside of cannula 723, and constructed so that it extends from the distal end of cannula 723. Typically, the introducer needle will be constructed to extend approximately 3 mm from the end of cannula 723, although it will be appreciated that other distances may be used. As the needle tab 721 is rotated around hinge 731, the needle 721 is inserted into and through the human tissue, and also the head 720 is moved toward the adhesive base 710.

View 702 shows the introducer needle 721 fully rotated towards the insertion point. In this way, the head 720 has been moved to the adhesive base 710, and the introducer needle 721 has fully penetrated the patient tissue, thereby enabling cannula 723 to be put in its initial depth and location. View 703 shows the introducer needle 721 retracted. Upon retraction, the introducer needle may be discarded, while the hinge mechanism and housing may be discarded or set aside for later reuse, within sanitary guidelines. In this way, the head 720 remains attached to the adhesive base 710 and the cannula 723 is at its initial depth and location, as illustrated in view 704. The insertion set 700 may now be connected to a medication source, such as an infusion pump, and medication effectively delivered to the initial tissue position and location.

The insertion set 700 has a reset mechanism 725 for repositioning cannula 723 after initial insertion. In operation, the reset mechanism 725 cooperates with a portion 724 of the head 722 effectuate the repositioning. For example, the portion 724 may be threadably adjustable within the reset mechanism 725. In this way, a patient, when therapeutic effect needs to be improved, can rotate the head 722 to move the cannula 723 further into the tissue, or alternatively to retract the cannula 723. In another example, the portion 724 may be constructed to snap at a second location into reset mechanism 725. In this way, when therapeutic effect needs to be increased, the patient simply presses the head 720 in a downward fashion, thereby causing the portion 724 to step into a new location into reset mechanism 725. This action would cause the cannula 723 to move to a new depth and position. In an alternate example, the patient may be able to lift the portion 724 away from the reset mechanism 725, thereby extracting the cannula 723 a few millimeters into a new position. It will be understood that the setting mechanism and cannula can be constructed and made to cooperate in a way that can change only the depth, change only the position, or change both the depth and position. It will also be understood that the infusion set can easily be modified to insert and reset a biological sensor, such as a sensor for glucose monitoring.

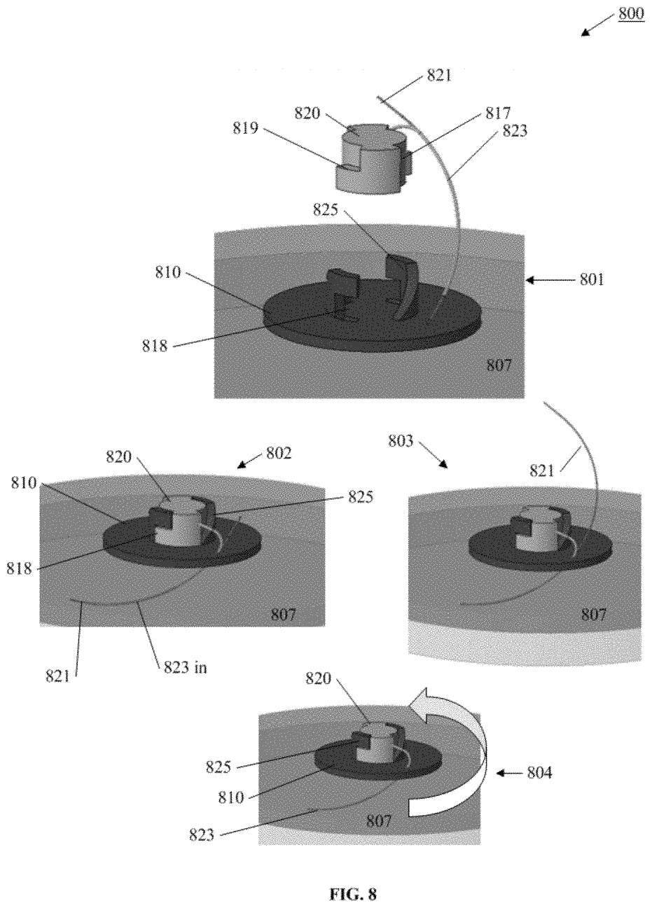

Referring now to FIG. 8, an infusion set 800 is illustrated. Infusion set 800 has an adhesive base 810 that secures against the skin of a patient 807. Infusion set 800 uses an insertion housing and mechanism (not shown) to position a cannula 823 at an initial depth and location. As illustrated, insertion set 800 uses an introducer needle to assist in initial positioning of cannula 820. As illustrated in view 801, a head piece 820 is mechanically moved by the housing into the adhesive base 810. In this way, the introducer needle 821 pierces and penetrates the tissue of the patient, and is used to position cannula 823 to its initial position and depth. In particular, the head piece 820 has a sloped portion 817 but cooperates with a sloped portion on the reset mechanism 825 to enable the headpiece 820 to be rotated and received into the reset mechanism 825. Rotation is complete when the tab portion of the head 820 is received into the tab portion 818 of the reset mechanism 825. As illustrated in view 802, the head portion 820 is fully rotated and received into the reset portion 825. In this way the tab 819 is fully received into the receiver tab 818. In this position, the introducer needle is extended fully into the patient tissue, and the cannula 823 is positioned at its initial depth and location.

As illustrated in view 803, the housing and any insertion mechanism may be removed, which also removes the introducer needle, all of which may be discarded. In this way, the head 820 remains in the reset mechanism 825. As illustrated in view 804, at a later time, when the patient desires to reset the cannula 823, the head 820 may be rotated within the reset mechanism 825 to retract cannula 823, thereby setting it into a new therapeutic position. In another example (not illustrated) the headpiece may initially be positioned not fully inserted into the reset mechanism, and then rotation of the headpiece by the patient can cause a further insertion of the cannula 823. In this latter case, the cannula tip would need to be hard enough or sharp enough to allow for a further insertion through patient tissue.

It will be understood that removal of the introducer needle may be accomplished in several ways. For example, as described above, the introduced needle may be removed as part of a manual motion by the patient to remove the insertion mechanism and housing. It will be appreciated that the introducer needle can be removed concurrent with removal of the housing or may be done prior to removing the housing. In one application, the introducer needle is removed by a spring or other tension device prior to removal of the housing. In this way, the removal of the introducer needle from the patient is independent of the action to remove the insertion mechanism and housing.

It will be understood that the setting mechanism and cannula of FIG. 8 can be constructed and made to cooperate in a way that can change only the depth, change only the position, or change both the depth and position. It will also be understood that the infusion set can easily be modified to insert and reset a biological sensor, such as a sensor for glucose monitoring.

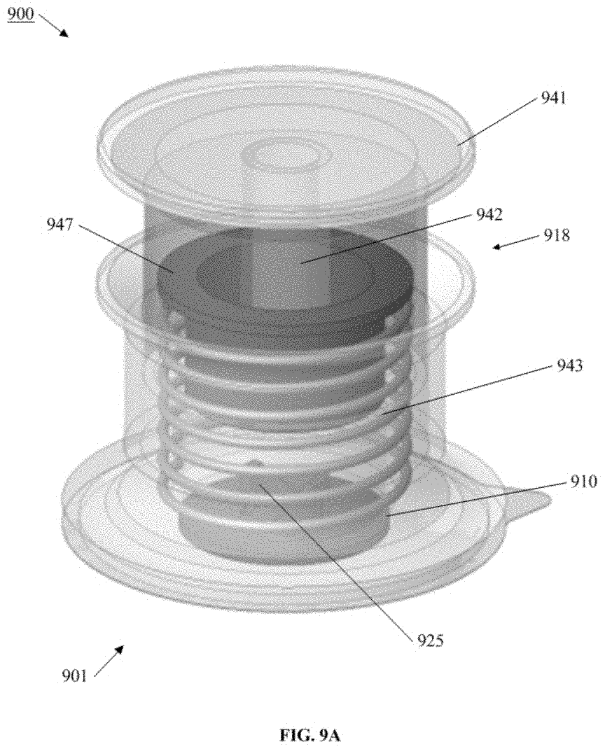

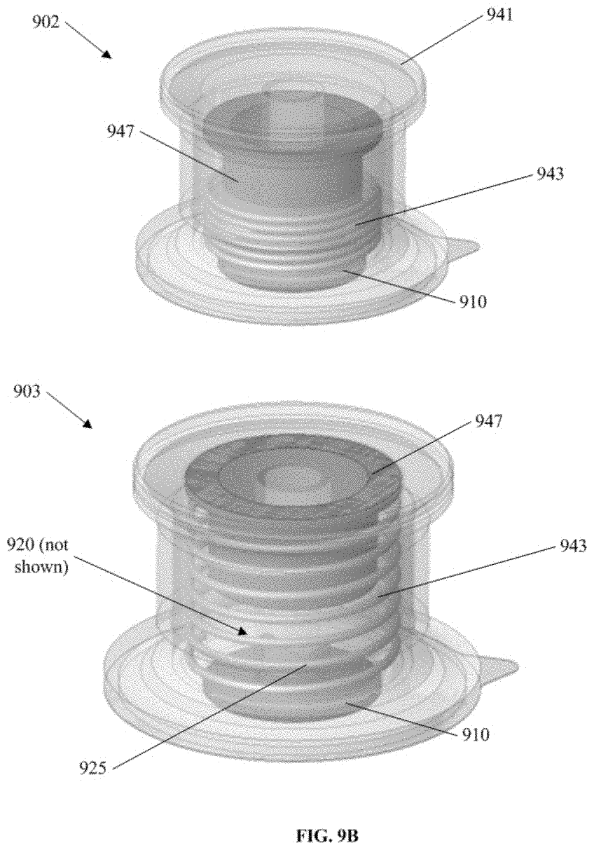

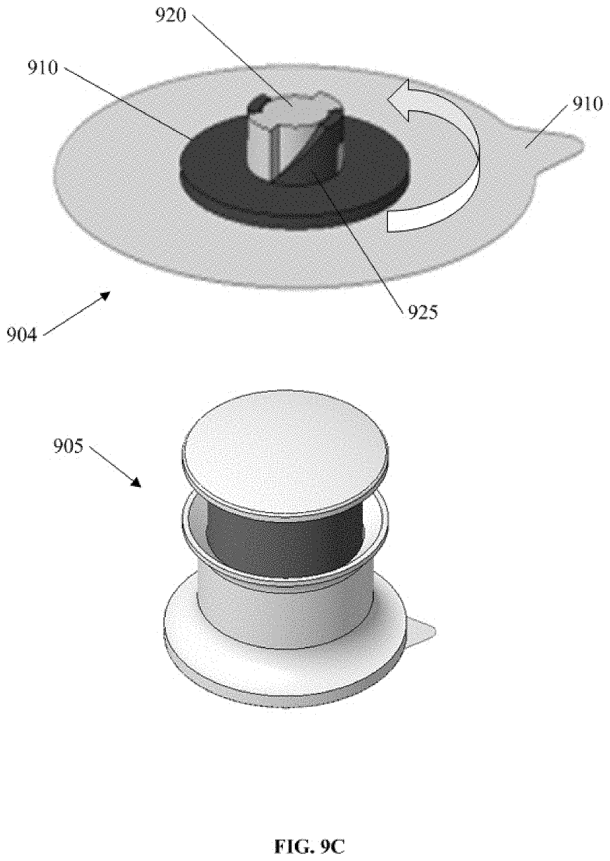

Referring now to FIGS. 9A, 9B and 9C, an infusion set 900 is illustrated. Infusion set 900 is one specific example of a housing and insertion mechanism that may be used to insert and initially position the infusion set 900 illustrated with respect to FIG. 8. It will be understood that there are a wide variety of insertion mechanisms and housings that may be used. Insertion set 900 has a housing 918 that has a tube piece 942 that initially holds the head piece 920. As illustrated in view 901, the headpiece 920 is retained securely in the tube 942 and is spaced away from the reset mechanism 925. In particular, the insertion mechanism 900 has a plunger 941 that is used by the patient to press the tube 942 through a sleeve 947 to move the head 920 toward the reset mechanism 925. As illustrated, a spring 943 is used to provide a force that initially keeps the plunger 941 fully extended away from the body of the patient. The housing 918 also has an adhesive pad which may be used to secure the infusion set 900 to the patient of the body.

View 902 shows that the patient has moved the plunger 941 fully towards the adhesive pad 910. As the patient moves plunger 941 such that the tube 943 moves downward in sleeve 947, the head 920 begins to engage the reset mechanism 925. Due to the curved and sloped nature of reset mechanism 925, the downward motion of the plunger 941 causes the head 920 to rotate clockwise and continue in a rotating downward motion until its tab locks into a mating tab of the reset mechanism 925, as described with reference to FIG. 8. It will be understood that the rotation direction can be set according the system requirements, for example, in some cases it may be useful to allow for a counterclockwise rotation to move the cannula downward. When the plunger 941 has been fully depressed by the patient, and the head 920 is firmly positioned in the reset mechanism 925, the patient may release the plunger 941, causing the spring 943 to expand, as illustrated in view 903. Alternatively, the spring maybe compressed to an actuation point which automatically engages the spring removal mechanism to retract the introducer needle from the body. As the spring expands, the introducer needle could then further be removed or even lockingly captured in the insertion mechanism. Further yet, expansion of the spring and removal of the insertion needle may cause the attachment between the insertion device and the housing base to be detached or decoupled, allowing for easy removal of the insertion device from the body and infusion site. For purposes of ease of illustration, the headpiece 920 is not shown in view 903. However, at this point the headpiece 920 would be firmly secured into the reset mechanism 925. The patient can now remove the housing 918 with all its associated setting mechanisms, including any introducer needle, which can be discarded.

As illustrated in view 904, with the housing removed, the head 920 remains securely attached into the reset mechanism 925, which is attached to the adhesive pad 910. The patient may now connect an infusion pump or other medicine insertion device to the head 920, and begin infusing medication into their body at the initial insertion depth and position. As previously described, the patient should obtain full therapeutic effect for a period of time, of at least 24 hours. At a later time, when therapeutic effect has been reduced at the initial site, the patient may reset the position of the cannula to obtain a new depth and location, thereby increasing therapeutic effect. As illustrated, the patient may rotate the head 920 in a counterclockwise direction to retract the cannula a few millimeters from the body. This places the cannula in a new therapeutic location, which will enable an extended and advantageous therapeutic effect for the medication. As described with reference to FIG. 8, the reset mechanism 925 may allow for one retraction, or may have multiple stops to allow for multiple retraction positions. Further, the insertion device and reset mechanism may be constructed such that the headpiece is initially positioned towards the top of the rotatable tabs, and thereby at a later time the patient would rotate the head in a clockwise position to insert the cannula further in to the tissue. View 905 shows the insertion set 900 in a solid view ready to be attached to the patient's skin. It will be understood that the setting mechanism and cannula of FIG. 9 can be constructed and made to cooperate in a way that can change only the depth, change only the position, or change both the depth and position. It will also be understood that the infusion set can easily be modified to insert and reset a biological sensor, such as a sensor for glucose monitoring.

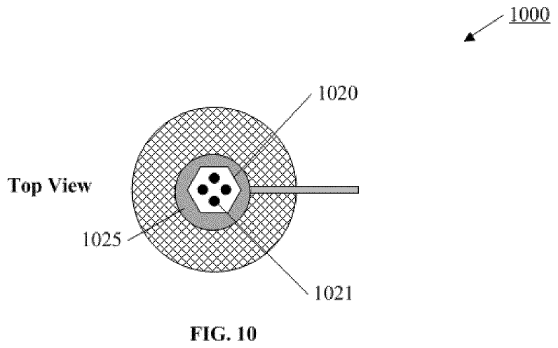

FIG. 10 shows an exemplary embodiment of an infusion set 1000 with multiple ports 1021 in the head 1020 to permit relocation of the straight, curved or helical needle in the same infusion set at the same site, in order to achieve new depots through new insertions. In some cases infusion set 1000 may have a reset mechanism 1025 as described with reference to FIG. 8, and in other cases the reset mechanism 1025 may also include an external setting device (not shown). For example, the patient may use a housing and setting tool to initially insert a cannula to a first depth and location as previously described. When the patient desires to set a new position and location, the patient removes the initially inserted cannula, and then inserts another cannula through one of the other ports 1021. In a alternative use, the patient may remove the cannula from a port, and insert a new cannula into the same port, and use the same insertion point through the skin. However, the new insertion would route to a new position and depth. For example, if the initial cannula went to the left of the port, the new insertion may be to the right of the port, thereby being in new tissue for infusion. In other examples, the new cannula can be selected to insert to an new depth. The initial cannula may be reusable in some circumstances, however, due to the risk of infection, it is more likely that old cannula would be discarded, and a new cannula inserted into a new port. The new cannula would be provided with an associated external setting device including a housing and insertion mechanism that would cooperate with the reset mechanism portion 1025 on the adhesive pad.

The patient would use the external setting device to insert the new cannula into one of the unused ports 1021, and to position the cannula to its therapeutic depth and position. In this way, the new cannula is set to a new depth and position as compared to the original cannula but reuses the same head piece 1020. The patient would then remove the external setting tool. In a alternative use, the patient may remove the cannula from a port, and insert a new cannula into the same port, and use the same insertion point through the skin. However, the new insertion would route to a new position and depth. For example, if the initial cannula went to the left of the port, the new insertion may be to the right of the port, thereby being located in new tissue for infusion. In other examples, the new cannula can be selected to insert to a new depth. Advantageously, the patient does not need to reset the adhesive pad and may use the same general location for a second insertion. Pain and discomfort is decreased, and the cost of inserting a new cannula is substantially reduced as compared to needing a full new insertion set. Additionally, the multiple ports would enable one of the ports to be used for a biological sensor, while another port could be used for infusion. Again, this increases patient comfort while reducing cost.

Although the headpieces are illustrated with 4 ports, it will be understood that more or fewer ports may be provided. For example, as briefly discussed above, a headpiece may be advantageously used having only a single port. That is, a single port can facilitate multiple cannula insertions over a period of time. For example, an initial cannula may be set by the patient through the single port to a particular position and location under the patient's skin. Upon degraded efficacy or a period of time, the patient may remove the first cannula, and insert a new cannula into the same port. In this way, the new cannula is inserted using the same existing insertion point through the skin, thereby reducing insertion force and pain or discomfort to the user. The cannula would be constructed or manipulated to be located at new position and depth different than the initial cannula. This may be accomplished, for example, by providing a set of cannulas, with each cannula designed to insert to a unique position and location. In another example, the set of cannulas may be similar, but have directional indicators that instruct the patient how to position and insert each cannula to assure a new position or depth.

While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

What is claimed is:

1. An extended use infusion set for a medical patient, comprising:
a cannula;
a head piece connected to the cannula, the head piece constructed to cooperate with a setting device to insert the cannula to an initial subdermal or subcutaneous depth and position; and
a reset mechanism operably connected to the cannula and constructed to reposition the cannula from its initial depth and position to a second depth and position.

2. The extended use infusion set according to claim 1, wherein the reset mechanism is coupled to the head piece.

3. The extended use infusion set according to claim 1, wherein the reset mechanism is movable by the patient in more than 1 dimension.

4. The extended use infusion set according to claim 1, wherein the head piece is constructed to rotate in the reset mechanism.

5. The extended use infusion set according to claim 1, wherein the head piece is constructed to slip towards or away from the reset mechanism.

6. The extended use infusion set according to claim 1, wherein the head piece is constructed to slide in tracks on the reset mechanism.

7. The extended use infusion set according to claim 1, wherein the reset mechanism is constructed to extend the cannula to set the second depth or position farther into the patient than the initial depth and position.

8. The extended use infusion set according to claim 1, wherein the reset mechanism is constructed to retract the cannula to set the second depth or position as compared to the initial depth and position.

9. The extended use infusion set according to claim 1, wherein the reset mechanism is operably connected to the cannula and constructed to reposition the cannula from its initial depth or position to one of a plurality of depths and positions.

10. The extended use infusion set according to claim 1, wherein the reset mechanism is operably connected to the cannula and constructed to reposition the cannula from its second depth and/or position to a third depth and position.

11. A method of using an extended use infusion set, comprising:
using a head piece to insert a cannula into an initial subdermal or subcutaneous depth and position;
securing the head piece to a patient;
delivering a medicine to the initial depth and position until efficacy of the medicine is below a threshold or the user desires to change the cannula's depth or position;
moving, using a reset mechanism, the cannula to a second subcutaneous depth and/or position; and
delivering the medicine to the second depth and/or position.

12. The method according to claim 11, further including:
delivering the medicine to the second depth and position until efficacy of the medicine is below a threshold or the user desires to change the cannula's depth or position;
moving, using a reset mechanism, the cannula to a third subcutaneous depth or position; and
delivering the medicine to the third depth or position.

13. The method according to claim 11, wherein the patient moves the reset mechanism in more than 1 dimension.

14. The method according to claim 11, wherein the patient rotates, slides, or snaps the head piece in the reset mechanism to effectuate moving the cannula into the second depth and/or position.

15. The method according to claim 11, wherein the cannula's subdermal or subcutaneous travel path to the second depth and/or position is longer as compared to the cannula's subcutaneous travel path to the initial depth and position.

16. The method according to claim 11, wherein the cannula's subdermal or subcutaneous travel path to the second depth and/or position is shorter as compared to the cannula's subcutaneous travel path to the initial depth and position.

17. An extended use infusion set for a medical patient, comprising:
a head piece having a plurality of cannula ports, the head piece removably attached to a patient's skin at an infusion site;
a reset mechanism further comprising:
a head piece portion attached to the head piece;
a setting tool removably attachable to the head piece portion; and
a cannula operably connected to the setting tool; and
wherein the patient connects the setting tool to the head piece portion and inserts the cannula through a used or an unused cannula port to position the cannula to a desired position and depth at the infusion site.

18. The extended infusion set according to claim 17, further including an introducer needle in the cannula.

19. The extended infusion set according to claim 17, further including a tube connected to the cannula and constructed to receive a medicine or therapeutic, or to contain a biosensor such as a glucose sensor.

20. The extended infusion set according to claim 17, further including:
a second setting tool removably attachable to the head piece portion;
a second cannula operably connected to the second setting tool; and
wherein the patient connects the second setting tool to the head piece portion and inserts the second cannula through the same used cannula port or another unused cannula port to position the second cannula to a desired second position and/or depth at the infusion site.

21. An extended use infusion set for a medical patient, comprising:
a head piece having a cannula port, the head piece removably attached to a patient's skin at an infusion site;
a first setting mechanism further comprising:
a head piece portion attached to the head piece;
a setting tool removably attachable to the head piece portion; and
a cannula operably connected to the setting tool; and
wherein the patient connects the setting tool to the head piece portion and inserts the cannula through the cannula port to position the cannula to a desired position and depth at the infusion site;
a second setting tool removably attachable to the head piece portion;
a second cannula operably connected to the second setting tool; and
wherein the patient connects the second setting tool to the head piece portion and inserts the second cannula through the cannula port to position the second cannula to a desired second position or depth at the infusion site.

22. The extended infusion set according to claim 21, wherein the first setting tool and the second setting tool are the same.

23. The extended infusion set according to claim 21, further including an introducer needle in the cannula.

24. The extended infusion set according to claim 21, further including a tube connected to the cannula and constructed to receive a medicine or therapeutic, or to contain a biosensor such as a glucose sensor.
